(19) United States
(12) Patent Application Publication
(10) Pub. No.: US 2023/0039844 A1
(43) Pub. Date: Feb. 9, 2023

(54) ADHESIVE REMOVER COMPOSITIONS

(71) Applicant: Kenya Latrice Dye, Chicago, IL (US)

(72) Inventor: Kenya Latrice Dye, Chicago, IL (US)

(21) Appl. No.: 17/871,041

(22) Filed: Jul. 22, 2022

Related U.S. Application Data
(60) Provisional application No. 63/224,953, filed on Jul. 23, 2021.

(51) Int. Cl. A61K 8/892; C11D 7/50; C11D 7/26; C11D 11/00; A61Q 5/02; A61Q 19/10; A61K 8/58

(52) U.S. Cl. CPC A61K 8/892; C11D 7/5022; C11D 7/261; C11D 11/0047; A61Q 5/02; A61Q 19/10; A61K 8/585

(57) ABSTRACT

Disclosed herein are adhesive remover compositions, methods of making such compositions, and methods of using such compositions. In one aspect, an adhesive remover composition comprises cyclopentasiloxane, dimethicone, and a blend or mixture of dimethicone and dimethiconol.

### RELATED APPLICATIONS

The present patent application claims the priority benefit of U.S. Provisional Patent Application Ser. No. 63/224,953, filed Jun. 23, 2021, the content of which is hereby incorporated by reference in its entirety into this disclosure.

### BACKGROUND

Topical adhesives such as surgical glue, electrode adhesive, and hair glue are commonly used for medical and cosmetic purposes. In the medical field, surgical glue, medical tape, and electrode adhesive are used to releasably couple medical equipment to the skin, hair, or scalp of a patient. Hair glue is frequently used in the cosmetic industry to bond natural or synthetic hair to the scalp and hair of an individual. These adhesives can be difficult to remove and leave unwanted residue on the skin and hair. The present disclosure relates to adhesive remover compositions that would benefit both the medical and cosmetic fields.

### BRIEF SUMMARY

In one aspect, the disclosure provides an adhesive remover composition comprising; a) cyclopentasiloxane; b) dimethicone; and c) a blend of dimethicone and dimethiconol.

In some aspects of the composition, the cyclopentasiloxane is in an inclusive range of about 72% w/w to about 88% w/w of the composition.

In some aspects of the composition, the dimethicone is in an inclusive range of about 16.5% w/w to about 20.5% w/w of the composition.

In some aspects of the composition, the blend of dimethicone and dimethiconol is in an inclusive range of about 1.3% w/w to about 1.7% w/w of the composition.

In some aspects of the composition, the composition is fluid at room temperature.

In another aspect, the disclosure provides a method for removing an adhesive from a subject in need thereof, the method comprising: applying a composition as disclosed herein to the adhesive on an external surface of the subject; and allowing sufficient time for the composition to loosen and/or release the adhesive from the external surface of the subject.

In some aspects of the method, the adhesive is an electrode or medical device adhesive.

In some aspects of the method, the adhesive is a hair bonding glue.

In some aspects of the method, the adhesive is a topical skin adhesive.

In some aspects of the method, the external surface of the subject is the skin, hair, and/or scalp.

In some aspects of the method, removing the loosened and/or released adhesive off of the skin comprises wiping or washing away the loosened and/or released adhesive.

In some aspects of the method, after applying the composition to the hair and/or scalp of the subject, the composition is massaged into the hair and/or scalp.

In some aspects of the method, removing the loosened and/or released adhesive comprises combing and/or shampooing the loosened and/or released adhesive out of the hair.

In yet another aspect, the disclosure provides a method for producing an adhesive remover composition, the method comprising: adding cyclopentasiloxane, dimethicone, and a blend of dimethicone and dimethiconol to a vessel; and mixing the cyclopentasiloxane, the dimethicone, and the blend of dimethicone and dimethiconol to achieve a uniform composition.

In some aspects of the method, the cyclopentasiloxane is in an inclusive range of about 72% w/w to about 88% w/w of the composition.

In some aspects of the method, the dimethicone is in an inclusive range of about 16.5% w/w to about 20.5% w/w of the composition.

In some aspects of the method, the blend of dimethicone and dimethiconol is in an inclusive range of about 1.3% w/w to about 1.7% w/w of the composition.

### DETAILED DESCRIPTION

Unless otherwise defined, all technical and scientific terms used herein have the same meaning as commonly understood by one of ordinary skill in the art to which the compositions described herein belong. The singular form “a”, “an” and “the” include plural referents unless the context clearly dictates otherwise. These articles refer to one or to more than one (i.e., to at least one). The term “and/or” means any one or more of the items in the list joined by “and/or”. As an example, “x and/or y” means any element of the three-element set {(x), (y), (x, y)}. In other words, “x and/or y” means “one or both of x and y”. As another example, “x, y, and/or z” means any element of the seven-element set {(x), (y), (z), (x, y), (x, z), (y, z), (x, y, z)}. In other words, “x, y and/or z” means “one or more of x, y and z”.

The term “about” as used in connection with a numerical value throughout the specification and the claims denotes an interval of accuracy, familiar and acceptable to a person skilled in the art. In general, such interval of accuracy is +/−10%.

Where ranges are given, endpoints are included. Furthermore, unless otherwise indicated or otherwise evident from the context and understanding of one of ordinary skill in the art, values that are expressed as ranges can assume any specific value or subrange within the stated ranges in different embodiments of the disclosure, to the tenth of the unit of the lower limit of the range, unless the context clearly dictates otherwise.

The term “exemplary” means serving as a non-limiting example, instance, or illustration. As utilized herein, the terms “e.g.,” and “for example” set off lists of one or more non-limiting aspects, examples, instances, or illustrations.

In a first aspect, the disclosure provides a composition for removing an adhesive (an adhesive remover composition). More specifically, the composition as described herein, can be applied to the hair, skin, and/or scalp of a subject to remove an adhesive or an adhesive residue. “Hair” as used herein generally refers to natural or synthetic hair on the head of a subject. However, the compositions and methods disclosed herein can also apply to body hair, facial hair, and eyelashes. When applied to a host surface (e.g., the hair, skin, and/or scalp of a subject), the composition creates a barrier between the host surface and the adhesive, which disrupts the bond between the adhesive and the host surface such that the adhesive is released from the host surface. Rather than dissolving an adhesive or adhesive residue, the composition disclosed herein has a “release and slide” effect, wherein the adhesive releases from a host surface in one or more pieces and then slides off the host surface for removal.

As used herein, the terms “subject” and “individual” are interchangeable and relate to vertebrates, preferably mammals. For example, mammals in the context of the disclosure are humans, non-human primates, domesticated animals such as dogs, cats, sheep, cattle, goats, pigs, horses, etc., laboratory animals such as mice, rats, rabbits, guinea pigs, etc., as well as animals in captivity such as animals in zoos. The term “animal” as used herein includes humans. The term “subject” can also refer to a patient undergoing a medical procedure in which a medical device or instrument is releasably coupled to the skin, hair, and/or scalp of a subject via an adhesive.

“Adhesive” as used herein refers to a substance that promotes adhesion (i.e., sticking together or attachment) between two or more surfaces. In the present disclosure, “adhesive”, “glue”, and “bond” can be used interchangeably. “Adhesive” is inclusive of adhesive residue. Adhesives of the present disclosure include, but are not limited to, topical skin adhesives, (also known as “medical skin glue”, “skin glue”, or “surgical glue”), electrode adhesives and gels, and hair adhesives (also known as “hair glue” or “hair bonding glue”). Exemplary topical skin adhesives include 2-Octyl-cyanoacrylate, n-2-Butyl-cyanoacrylate, and 2-Ethyl-cyanoacrylate. Hair adhesives include wig glue, weave glue, hair extension glue, and the like. Adhesive residues of the present disclosure include residue from topical skin adhesives, electrode adhesives and gels, hair adhesives, residue from adhesive bandages such as those used as wound or surgical dressings (e.g., hydrocolloid dressings and acrylic- and rubber-based adhesives), and residue from medical tape.

In some aspects, the composition is a serum, liquid, or solution comprising cyclopentasiloxane, dimethicone, and a blend or mixture of dimethicone and dimethiconol (the dimethicone of the blend or mixture being distinct from the dimethicone as an independent ingredient). In certain aspects, the composition consists of or consists essentially of cyclopentasiloxane, dimethicone, and a blend or mixture of dimethicone and dimethiconol.

In some aspects of the composition, the cyclopentasiloxane is in an inclusive range of about 72% w/w to about 88% w/w of the composition. In some aspects, the cyclopentasiloxane is about 72%, about 73%, about 74%, about 75%, about 76%, about 77%, about 78%, about 79%, about 80%, about 81%, about 82%, about 83%, about 84%, about 85%, about 86%, about 87%, or about 88% w/w of the composition.

In some aspects of the composition, the dimethicone is in an inclusive range of about 16.5% w/w to about 20.5% w/w of the composition. In some aspects, the dimethicone is about 16.5%, about 17%, about 17.5%, about 18%, about 18.5%, about 19%, about 19.5%, about 20%, or about 20.5% w/w of the composition.

In some aspects of the composition, the blend of dimethicone and dimethiconol is in an inclusive range of about 1.3% w/w to about 1.7% w/w of the composition. In some aspects, the blend of dimethicone and dimethiconol is about 1.3%, about 1.4%, about 1.5%, about 1.6%, or about 1.7% w/w of the composition.

In some aspects, the composition is fluid and flowable at room temperature.

In some aspects, the composition can comprise additional components such as water, dyes, fragrances, essential oils, aloe or aloe extract, alcohol, propylene glycol methyl ether, dipropylene glycol methyl ether, isopropyl myristate, isoparaffin, and other additives and/or solvents. In some aspects, the composition can comprise medical or industrial grade carriers or diluents that would be known to one of ordinary skill.

In another aspect, the disclosure provides a method for removing an adhesive from a subject, the method comprising: applying a composition as disclosed herein to the subject; and allowing sufficient time for the composition to loosen and/or release the adhesive from the subject. Exemplary adhesives of the method include electrode adhesives, hair bonding glue, and topical skin adhesives.

Medical adhesive-related skin injuries can occur when the attachment between the skin and an adhesive is stronger than that between individual cells, causing either the epidermal layers to separate or the epidermis to detach completely from the dermis. The “release and slide” effect of the claimed compositions are particually benefical for patients requiring adhesive medical devices who are at high risk of infection.

In particular aspects, the method can be used to remove electrode glue from a patient undergoing a medical procedure requiring placement of electrodes on the skin, hair, or scalp (e.g., electrocardiogram, electronystagmography, polysomnogram, or electromyography). Such a method can include: applying a composition as disclosed herein to the skin, hair, and/or scalp of a subject; allowing sufficient time for the composition to loosen and/or release the electrode glue from the subject; removing the loosened and/or released electrode glue from the skin, hair, and/or scalp. In some aspects, after application, the composition can be massaged into the skin, hair, and/or scalp of the patient. In some aspects, the loosened and/or released electrode glue can be wiped away and/or washed off the skin, hair, and/or scalp.

In particular aspects, the method can be used to remove topical skin adhesive from a patient undergoing or recovering from a medical procedure. Such a method can include: applying a composition as disclosed herein to the skin, hair, and/or scalp of a subject; allowing sufficient time for the composition to loosen and/or release the topical skin adhesive from the subject; removing the loosened and/or released topical skin adhesive from the skin, hair, and/or scalp. In some aspects, after application, the composition can be massaged into the skin, hair, and/or scalp of the patient. In some aspects, the loosened and/or released electrode glue can be wiped away and/or washed off the skin, hair, and/or scalp. In some aspects, the method is implemented to remove topical skin adhesive, or adhesive residue, from a bandage, wound or surgical dressing, or from a medical device or instrument that was releasably coupled to the skin, hair, and/or scalp of the patient during a medical procedure. For example, residue from medical tape used to hold an IV line in place.

In particular aspects, the method can be used to remove hair glue from a subject. Such a method can include: applying a composition as disclosed herein to the hair and/or scalp of a subject; allowing sufficient time for the composition to loosen and/or release the hair glue from the hair and/or scalp of the subject; and removing the loosened and/or released hair glue from the hair and/or scalp. In some aspects, after application, the composition can be massaged into the hair and/or scalp of the subject. In some aspects, the loosened and/or released hair glue can be removed by combing and/or shampooing the treated hair.

In some aspects of the methods disclosed herein, the composition is applied directly to and around the area (i.e., the hair, skin, or scalp of a subject) for which adhesive or adhesive residue removal is desired.

In some aspects, the body heat of the subject can aid in, but is not required for, the “release and slide” effect of the composition. In some aspects, if heating of the composition is desired, an external heat source can be applied to the area. If desired, heating the composition can result in a thinner or more fluid consistency.

In yet another aspect, the disclosure provides a method for producing an adhesive remover composition, the method comprising: adding cyclopentasiloxane, dimethicone, and a blend of dimethicone and dimethiconol to a vessel; and mixing the cyclopentasiloxane, the dimethicone, and the blend of dimethicone and dimethiconol to achieve a uniform composition. The ingredients can be added in any order.

In some aspects of the method, the cyclopentasiloxane is in an inclusive range of about 72% w/w to about 88% w/w of the composition. In some aspects, the cyclopentasiloxane is about 72%, about 73%, about 74%, about 75%, about 76%, about 77%, about 78%, about 79%, about 80%, about 81%, about 82%, about 83%, about 84%, about 85%, about 86%, about 87%, or about 88% w/w of the composition.

In some aspects of the method, the dimethicone is in an inclusive range of about 16.5% w/w to about 20.5% w/w of the composition. In some aspects, the dimethicone is about 16.5%, about 17%, about 17.5%, about 18%, about 18.5%, about 19%, about 19.5%, about 20%, or about 20.5% w/w of the composition.

In some aspects of the method, the blend of dimethicone and dimethiconol is in an inclusive range of about 1.3% w/w to about 1.7% w/w of the composition. In some aspects, the blend of dimethicone and dimethiconol is about 1.3%, about 1.4%, about 1.5%, about 1.6%, or about 1.7% w/w of the composition.

In some aspects, the method is performed at room temperature.

In some aspects, equipment used to produce the adhesive remover composition are dry and free from moisture.

### EXAMPLES

### Adhesive Remover Composition

Table 1 illustrates an adhesive and bond remover composition according to some aspects of the present disclosure.

TABLE 1
Adhesive Remover Composition

| International | | |
|---|---|---|
| Nomenclature | | |
| Cosmetic Ingredient | | |
| (INCI) | Weight % (w/w) | Supplier |
| Cyclopentasiloxane | 80 | ShinEtsu, KF995 |
| Dimethicone | 18.5 | GZ QIFU, QF-50W |
| Dimethicone and | 1.5 | Dow Corning, DC 1403 |
| Dimethiconol | | |

The ingredients of Table 1 were added to a mixing vessel and mixed until a uniform composition (e.g., even distribution of ingredients) was achieved.

Adhesive Product Removal from Skin

The edges of an adhesive product on a patient were loosened. The adhesive remover composition was applied to the exposed skin. The adhesive was held down with one hand and with the fingers of the opposite hand, the skin was pushed down and away from the adhesive. The adhesive product was gently removed, keepning it as close to the skin as possible and more of the adhesive remover composition was applied to the skin as the adhesive product begins to lift. After removal of the adhesive product, the adhesive remover composition was applied to the skin of the patient and allowed to rest on the remaining adhesive for 5-10 seconds. The skin was then gently wiped to remove the remaining traces of adhesive.

Adhesive Removal from Hair

The adhesive remover composition was applied to the hair of a patient. The adhesive remover composition was massaged into the patient's hair and allowed to sit for 30-60 seconds. After the allotted time passed, the remaining adhesive was wiped out of the patient's hair. The patient's hair was then combed and shampooed.

The presently described technology is now described in such full, clear, concise, and exact terms as to enable any person skilled in the art to which it pertains, to practice the same. It is to be understood that the foregoing describes preferred aspects of the technology and that modifications may be made therein without departing from the spirit or scope of the invention as set forth in the appended claims.

1. An adhesive remover composition comprising:
a) cyclopentasiloxane;
b) dimethicone; and
c) a blend of dimethicone and dimethiconol.

2. The composition of claim 1, wherein the cyclopentasiloxane is in an inclusive range of about 72% w/w to about 88% w/w of the composition.

3. The composition of claim 1, wherein the dimethicone is in an inclusive range of about 16.5% w/w to about 20.5% w/w of the composition.

4. The composition of claim 1, wherein the blend of dimethicone and dimethiconol is in an inclusive range of about 1.3% w/w to about 1.7% w/w of the composition.

5. The composition of claim 1, wherein the composition is fluid at room temperature.

6. A method for removing an adhesive from a subject in need thereof, the method comprising:
a) applying the composition of claim 1 to the adhesive on an external surface of the subject; and
b) allowing sufficient time for the composition to loosen and/or release the adhesive from the external surface of the subject.

7. The method of claim 6, wherein the adhesive is an electrode adhesive.

8. The method of claim 6, wherein the adhesive is a hair bonding glue.

9. The method of claim 6, wherein the adhesive is a topical skin adhesive.

10. The method of claim 6, wherein the external surface of the subject is skin.

11. The method of claim 10, wherein removing the loosened and/or released adhesive off of the skin comprises wiping or washing away the loosened and/or released adhesive.

12. The method of claim 6, wherein the external surface of the subject is hair and/or scalp.

13. The method of claim 12, further comprising massaging the composition into the hair and/or scalp of the subject after a).

14. The method of claim 12, wherein removing the loosened and/or released adhesive comprises combing and/or shampooing the loosened and/or released adhesive out of the hair.

15. A method for producing an adhesive remover composition, the method comprising:
a) adding cyclopentasiloxane, dimethicone, and a blend of dimethicone and dimethiconol to a vessel; and
b) mixing the cyclopentasiloxane, the dimethicone, and the blend of dimethicone and dimethiconol to achieve a uniform composition.

16. The method of claim 15, wherein the cyclopentasiloxane is in an inclusive range of about 72% w/w to about 88% w/w of the composition.

17. The method of claim 15, wherein the dimethicone is in an inclusive range of about 16.5% w/w to about 20.5% w/w of the composition.

18. The method of claim 15, wherein the blend of dimethicone and dimethiconol is in an inclusive range of about 1.3% w/w to about 1.7% w/w of the composition.
